(19) United States
(12) Patent Application Publication
(10) Pub. No.: US 2022/0054855 A1
(43) Pub. Date: Feb. 24, 2022

(54) TREATMENT SUPPORT SYSTEM AND TREATMENT SUPPORT DEVICE

(71) Applicant: Shimadzu Corporation, Kyoto (JP)

(72) Inventor: Akihiro ISHIKAWA, Kyoto (JP)

(21) Appl. No.: 17/370,780

(22) Filed: Jul. 8, 2021

(30) Foreign Application Priority Data
Aug. 24, 2020 (JP) 2020-141049

(51) Int. Cl. A61N 5/06

(52) U.S. Cl. CPC A61N 5/062

(57) ABSTRACT

A treatment support system is provided with an irradiation unit for irradiating a medical agent containing a fluorescent material with treatment light, a light detection unit for detecting, out of the treatment light and fluorescence, at least the fluorescence, a storage uni, and a control unit. The control unit performs control of at least a start of storing the fluorescence signal by the storage unit based on that the fluorescence signal detected by the light detection unit has changed beyond at least a predetermined degree of change; or that the light detection unit has detected the signal of the treatment light.

### CROSS-REFERENCE TO RELATED APPLICATIONS

The priority application number JP2020-141049, entitled “TREATMENT SUPPORT SYSTEM AND TREATMENT SUPPORT DEVICE”, filed on Aug. 24, 2020, invented by Akihiro Ishikawa, upon which this patent application is based, is hereby incorporated by reference in its entirety.

### BACKGROUND OF THE INVENTION

### Field of the Invention

The present invention relates to a treatment support system and a treatment support device.

### Description of the Background Art

Conventionally, a treatment support device is known in which a support of treatment (photoimmunotherapy) for killing cancer cells by emitting treatment light in a predetermined wavelength range to a medical agent containing a fluorescent material administered in a body of a subject or the treatment is performed. Such a treatment support device is disclosed in WO 2019/215905.

WO 2019/215905 discloses a treatment support device provided with a fluorescence detection unit for detecting fluorescence and a fluorescence image generation unit for generating a fluorescent image based on a fluorescence signal output by the fluorescence detection unit. The fluorescence detection unit detects the fluorescence emitted by the fluorescent material of the medical agent administered into a body of a subject for treatment by photoimmunotherapy. The treatment support device described in WO 2019/215905 is configured to output a fluorescent image generated by the fluorescence image generation unit before the treatment and a fluorescence image generated by the fluorescence image generation unit at the time of the treatment.

Using the treatment support device described in WO 2019/215905, a user, such as, e.g., a doctor, confirms the progress of the treatment for a cancer cell based on the detected change in the fluorescence by comparing the fluorescent image before the treatment with the fluorescent image at the time of the treatment.

Here, although not specifically described in WO 2019/215905, in a conventional treatment support device as described in WO 2019/215905, in order to confirm the progress of treatment by photoimmunotherapy, the change in the fluorescence signal is acquired together with the fluorescent image. In this instance, since a user, such as, e.g., a doctor, performs an operation of starting irradiation of treatment light and then performs an operation of recording a fluorescence signal in order to acquire the change in the fluorescence signal, a time difference occurs between the start time of the irradiation of the treatment light and the start time of the recording of the fluorescence signal. For this reason, there is a need for a treatment support system and a treatment support device capable of starting the storage of the fluorescence signal without a time lag at the timing of the treatment start.

The present invention has been made to solve the above-described problems. One object of the present invention is to provide a treatment support system and a treatment support device capable of performing a start of storing a fluorescence signal without a time difference at the timing of the treatment start.

### SUMMARY OF THE INVENTION

A treatment support system according to a first aspect of the present invention includes:

an irradiation unit configured, in treatment of killing a cancer cell by irradiating a medical agent containing a fluorescent material that has been administered into a body of a subject with treatment light in a predetermined wavelength range, to irradiate the medical agent with the treatment light;

a light detection unit configured to detect, out of the treatment light and fluorescence emitted by the fluorescent material of the medical agent excited by irradiation of the treatment light, at least the fluorescence emitted by the fluorescent material of the medical agent excited by the irradiation of the treatment light;

a storage unit configured to store a fluorescence signal of the fluorescence detected by the light detection unit; and

a control unit configured to perform control of at least a start of storing the fluorescent signal by the storage unit,

wherein the control unit is configured to

include a change determination unit for determining whether or not the fluorescence signal detected by the light detection unit has changed beyond at least a predetermined degree of change and a first storage control unit for performing control of at least the start of storing the fluorescence signal by the storage unit when the change determination unit determines that the fluorescence signal detected by the light detection unit has changed beyond at least the predetermined degree of change, and

perform control of at least the start of storing the fluorescence signal by the storage unit based on that the fluorescence signal detected by the light detection unit has changed beyond at least the predetermined degree of change, or

wherein the control unit is configured to

include a treatment light determination unit for determining whether or not the light detection unit has detected a signal of the treatment light and a second storage control unit for performing control of the start of storing the fluorescence signal by the storage unit when the treatment light determination unit determines that the light detection unit has detected the signal of the treatment light, and

perform control of at least the start of storing the fluorescence signal by the storage unit based on that the light detection unit has detected the signal of the treatment light.

A treatment support device according to a second aspect of the present invention includes:

a light detection unit configured, in treatment of killing a cancer cell by irradiating a medical agent containing a fluorescent material that has been administered into a body of a subject with treatment light in a predetermined wavelength range, to detect, out of the treatment light and the fluorescence emitted by the fluorescent material of the medical agent excited by irradiation of the treatment light, at least the fluorescence emitted by the fluorescent material of the medical agent excited by the irradiation of the treatment light;

a storage unit configured to store a fluorescence signal of the fluorescence detected by the light detection unit; and

a control unit configured to perform control of at least a start of storing the fluorescent signal by the storage unit,

wherein the control unit is configured to

include a change determination unit for determining whether or not the fluorescence signal detected by the light detection unit has changed beyond at least a predetermined degree of change and a first storage control unit for performing control of at least the start of storing the fluorescence by the storage unit when the change determination unit determines that the fluorescence signal detected by the light detection unit has changed beyond by at least the predetermined degree of change, and

perform control of at least the start of storing the fluorescence signal by the storage unit based on that the fluorescence signal detected by the light detection unit has changed beyond at least the predetermined degree of change, or

wherein the control unit is configured to

include a treatment light determination unit for determining whether or not the light detection unit has detected a signal of the treatment light and a second storage control unit for performing control of the start of storing the fluorescence signal by the storage unit when the treatment light determination unit determines that the light detection unit has detected the signal of the treatment light, and

perform control of at least the start of storing the fluorescence signal by the storage unit based on that the light detection unit has detected the signal of the treatment light.

Here, when the irradiation of treatment light is started and the treatment is started, the fluorescent material contained in the medical agent used for the treatment is excited, and fluorescence is emitted from the fluorescent material contained in the medical agent. Therefore, when the treatment light radiation is started and the treatment is started, the strength of the fluorescence signal changes abruptly.

In the treatment support system according to the first aspect of the present invention and the treatment support device according to the second aspect thereof, in a case where it is configured to control at least the start of storing the fluorescence signal by the storage unit, based on that the fluorescence signal detected by the light detection unit has changed beyond at least a predetermined degree of change, the control unit can acquire the timing of the treatment start, based on that the fluorescence signal detected by the light detection unit has changed beyond at least the predetermined degree of change. Therefore, the control of the start of storing the fluorescence signal by the storage unit can be performed at the timing of the treatment start without a time lag. As a result, it is possible to provide a treatment support system and a treatment support device capable of performing the start of storing the fluorescence signal without a time lag at the timing of the treatment start.

Since the treatment is started by performing the irradiation of the treatment light with the irradiation unit, the signal of the treatment light is detected at the time of starting the treatment. In the treatment support system according to the first aspect of the present invention and the treatment support device according to the second aspect thereof, in a case where it is configured to perform control of at least the start of storing the fluorescence signal by the storage unit based on that the light detection unit has detected the signal of the treatment light, the control unit can acquire the timing of the treatment start based on that the light detection unit has detected the signal of the treatment light. Therefore, the control of the start of storing the fluorescence signal by the storage unit can be performed at the timing of the treatment start without a time lag. As a result, it is possible to provide a treatment support system and a treatment support device capable of performing the start of storing the fluorescence signal without a time lag at the timing of the treatment start.

### BRIEF DESCRIPTION OF THE DRAWINGS

FIG. 1 is a block diagram showing the overall configuration of a treatment support system according to a first embodiment of the present invention.

FIG. 2 is a diagram showing an example of a fluorescent image according to the first embodiment.

FIG. 3 is a diagram showing an example of a treatment light image according to the first embodiment.

FIG. 4 is a diagram showing an example of an image showing a change in the fluorescence signal strength.

FIG. 5 is a diagram showing an example of a composite image according to the first embodiment.

FIG. 6 is a diagram showing an example of a change in the fluorescence signal strength from before the treatment start to after the treatment start.

FIG. 7 is a diagram showing an example of a change in the differential signal of the fluorescence signal strength from before the treatment start to after the treatment start.

FIG. 8 is a block diagram showing the overall configuration of a treatment support system according to the second embodiment of the present invention.

FIG. 9 is a block diagram showing the overall configuration of a treatment support system according to a third embodiment of the present invention.

FIG. 10 is a block diagram showing the overall configuration of a treatment support system according to a first modification of the present invention.

### DESCRIPTION OF THE PREFERRED EMBODIMENT

Hereinafter, embodiments in which the present invention is embodied will be described with reference to the attached drawings.

### First Embodiment

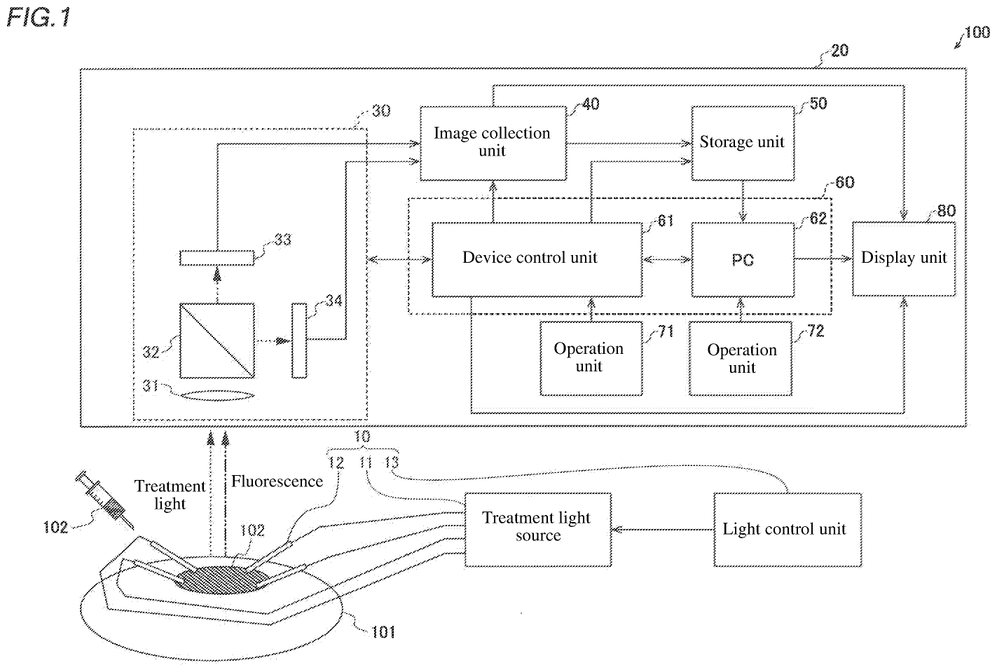

Referring to FIG. 1 to FIG. 7, the configuration of a treatment support system 100 according to a first embodiment will be described.

The treatment support system 100 according to this embodiment is a system for supporting treatment in photoimmunotherapy. Specifically, as shown in FIG. 1, the treatment support system 100 is configured to emit treatment light (excitation light) to a cancer patient 101 to detect fluorescence emitted from a fluorescent material of a medical agent 102 administered into a body of the cancer patient 101. Further, in addition to the support of treatment by photoimmunotherapy, the treatment support system 100 is configured to perform treatment by photoimmunotherapy continuously emitting treatment light, which is light in a certain wavelength range, according to the fluorescent material of the medical agent 102. Note that the cancer patient 101 is an example of the “subject” recited in claims. The cancer patient 101 may be targeted to animals other than humans.

As shown in FIG. 1, the treatment support system 100 includes an irradiation unit 10 and a treatment support device 20. In the treatment support system 100, the irradiation unit 10 and the treatment support device 20 (light detection unit 30) are provided separately and independently. The irradiation unit 10 and the treatment support device 20 (light detection unit 30) are controlled by a light control unit 13 and a control unit 60, respectively, as described below.

### (Configuration of Irradiation Unit)

The irradiation unit 10 is configured to irradiate the medical agent 102 with treatment light in the treatment of killing cancer cells based on the irradiation of treatment light in a predetermined wavelength range to the medical agent 102 containing a fluorescent material administered into the body of a cancer patient 101. That is, the irradiation unit 10 is configured to emit the treatment light (excitation light) to the medical agent 102 containing the fluorescent material administered into the body of the cancer patient 101 in the treatment by the photoimmunotherapy. As shown in FIG. 1, the irradiation unit 10 includes a treatment light source 11, a plurality of treatment probes 12, and a light control unit 13.

The treatment light source 11 is configured to emit the treatment light (excitation light) in a specific wavelength range for exciting the fluorescent material contained in the medical agent 102. The treatment light source 11 includes a laser diode (LD: Laser Diode), a light-emitting diode (LED: Light Emitting Diode), or the like.

The treatment probe 12 is configured to be inserted into the body of the cancer patient 101 to emit the treatment light within the body of the cancer patient 101. The light control unit 13 is configured to perform control of turning on and off the treatment light source 11 (emitting the treatment light and stop of emitting the treatment light). In the first embodiment, the light control unit 13 is provided independently of the control unit 60 (device control unit 61) of the treatment support device 20. Further, the light control unit 13 is configured to perform control of a start of emitting the treatment light and a stop tof emitting the treatment light (ON/OFF switching of the treatment light emission) in accordance with the operation of a user, such as, e.g., a doctor, operating an operation unit (not shown).

The treatment probe 12 includes an optical fiber for guiding the light from the treatment light source 11. The treatment probe 12 is inserted along a cylindrical guide (not shown), such as, e.g., a diffuser, made of a transparent material, such as, e.g., glass, to be inserted into the body of the cancer patient 101 toward the position (treatment site) that is a treatment site in the body of the cancer patient 101.

A user, such as, e.g., a doctor, grasps the position of the cancer in advance using an MRI (Magnetic Resonance Image), an X-ray CT (Computed Tomography), an ultrasonic wave echo, or the like. Then, the user, such as, e.g., a doctor, inserts the treatment probe 12 into the body of the cancer patient 101 while confirming the position of the cancer by an ultrasonic wave echo or the like. The treatment probe 12 is configured to guide and emit the treatment light from the treatment light source 11 in the body of the cancer patient 101. With this, the fluorescent material of the medical agent 102 is excited by the treatment light.

The treatment support system 100 can perform the treatment (photoimmunotherapy) for killing cancer cells by continuously emitting the treatment light, which is light in a particular wavelength range for exciting the fluorescent material contained in the medical agent 102, in the body of the cancer patient 101 with the treatment probe 12.

In photoimmunotherapy, the medical agent 102 (see FIG. 1) is administered into the body of a cancer patient 101 (see FIG. 1) before emitting the treatment light. The medical agent 102 contains a fluorescent material that emits fluorescence and an antibody. The fluorescent material of the medical agent 102 is a substance that is excited to emit fluorescence by being irradiated with treatment light and a substance that causes a photochemical reaction by being continuously irradiated with treatment light. The fluorescent material is a chemical substance, such as, e.g., an IRDye (registered mark) 700DX.

At the time of treatment by photoimmunotherapy, the irradiation unit 10 emits treatment light corresponding to the type of the fluorescent material of the medical agent 102 administered to the cancer patient 101 to the treatment site (cancer cell) of the cancer patient 101.

Note that the treatment light to be irradiated by the irradiation unit 10 at the time of treatment is light in a wavelength range in which the fluorescent material of the medical agent 102 used for the treatment causes a photochemical reaction in a wavelength region of 600 nm or more and 2,500 nm or less, which is a wavelength region from a part of visible light to near-infrared light, and differs depending on the type of the fluorescent material of the medical agent 102 used for the treatment. For example, in a case where the IRDye 700DX is used for the fluorescent material of the medical agent 102, the irradiation unit 10 irradiates light having a peak position of a wavelength of 600 nm or more and 700 nm or less, for example, non-thermal red light (near-infrared light) having a peak position of a wavelength of about 690 nm during the treatment by the photoimmunotherapy

### (Configuration of Treatment Support Device)

As shown in FIG. 1, the treatment support device 20 of the treatment support system 100 includes a light detection unit 30, an image collection unit 40, and a storage unit 50. As shown in FIG. 1, the treatment support device 20 of the treatment support system 100 is provided with a control unit 60, an operation unit 71, an operation unit 72, and a display unit 80.

The light detection unit 30 is configured to detect, out of the treatment light and the fluorescence emitted by the fluorescent material of the medical agent 102 excited by the irradiation of the treatment light, at least the fluorescence emitted by the fluorescent material of the medical agent 102 excited by the irradiation of the treatment light.

As shown in FIG. 1, the light detection unit 30 of the treatment support system 100 includes a lens 31 and a prism 32. Further, the light detection unit 30 of the treatment support system 100 includes a fluorescence detection unit 33 and a treatment light detection unit 34.

The lens 31 is configured such that the fluorescence emitted by the fluorescent material of the medical agent 102 and the visible light containing the treatment light emitted by the irradiation unit 10 are incident. The fluorescence and the visible light including the treatment light, which are incident on the lens 31, are converged by the lens 31 and incident on the prism 32.

The prism 32 is configured to separate the incident light, and the fluorescence and the visible light including the treatment light, which are incident on the lens 31, are separated by the prism 32. The fluorescence separated by the prism 32 is configured to be imaged at the fluorescence detection unit 33. The visible light containing the treatment light separated by the prism 32 is configured to be imaged at the treatment light detection unit 34.

The fluorescence detection unit 33 is configured to detect the fluorescence emitted by the fluorescent material of the medical agent 102 excited by the irradiation of the treatment light. The fluorescence detection unit 33 includes an image sensor for capturing an image based on the fluorescence emitted by the fluorescent material of the medical agent 102 separated by the prism 32. The image sensor photoelectrically converts the fluorescence into an electric signal. The image sensor is, for example, a CMOS (Complementary Metal Oxide Semiconductor) image sensor, or a CCD (Charge Coupled Device) image sensor.

In other words, the fluorescence detection unit 33 is configured to detect the fluorescence emitted by the fluorescent material of the medical agent 102 excited by the irradiation of the treatment light and acquire (capture) the fluorescent image 81 (see FIG. 2) based on the detected fluorescence. Note that the fluorescent image 81 is an image representing the distribution state of the fluorescence emitted by the fluorescent material of the medical agent 102.

The fluorescence detection unit 33 is configured to selectively detect the light in the region including the wavelength range of the fluorescence emitted by the fluorescent material of the medical agent 102 by the wavelength-selectivity of the optical filter. For example, in a case where an IRDye 700DX is used for the fluorescent material of the medical agent 102, the fluorescence detection unit 33 is configured to detect the fluorescence and capture the fluorescent image 81 based on the light having a wavelength of 700 nm or more by the wavelength-selectivity of the optical filter. Note that the IRDye 700DX is excited by the light having a wavelength of 600 nm or more and 700 nm or less, and emits the light having a peak at a wavelength of about 700 nm or 770 nm as fluorescence.

The treatment light detection unit 34 is configured to detect the treatment light. The treatment light detection unit 34 includes an image sensor that detects the visible light containing the treatment light separated by the prism 32 and captures the image based on the visible light containing the detected treatment light. The image sensor photoelectrically converts the visible light including the treatment light into an electric signal. The image sensor is, for example, a CMOS image sensor, a CCD image sensor, or the like.

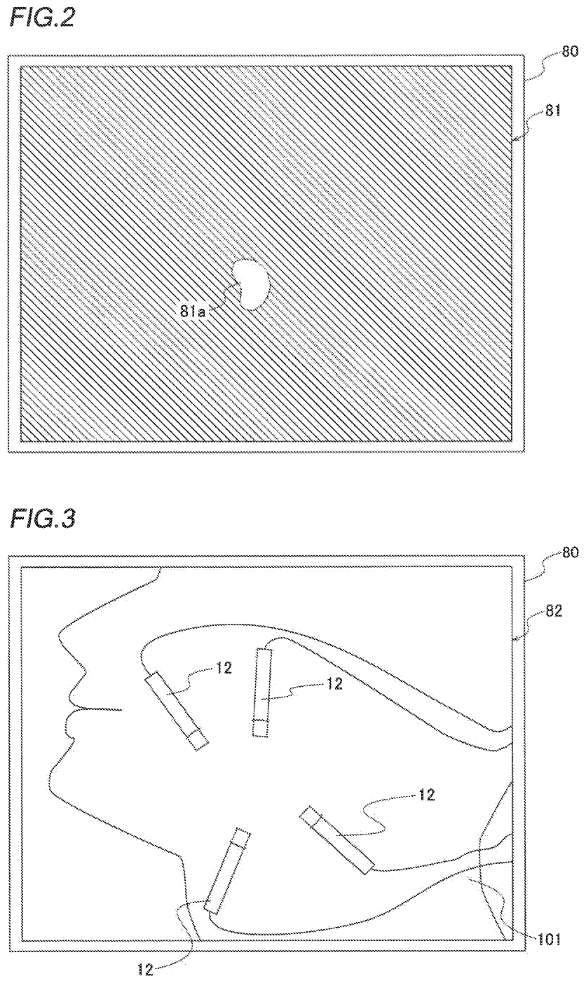

The treatment light detection unit 34 is configured to selectively detect the light in the region including the wavelength range of the treatment light emitted by the irradiation unit 10 (treatment probe 12) by the wavelength-selectivity of the optical filter. In a case where the light having a wavelength of 600 nm or more and 700 nm or less, for example, non-thermal red light having a wavelength peak position of about 690 nm, is emitted at the time of the treatment by photoimmunotherapy, the treatment light detection unit 34 is configured to detect the visible light including the treatment light based on the light having a wavelength of 400 nm or more and 700 nm or less including a wavelength range of treatment light and a wavelength range of visible light by the wavelength-selectivity of the optical filter, and capture the treatment light image 82 (see FIG. 3). That is, the treatment light image 82 includes a visible light image. Further, the treatment light image 82 is a color image captured based on the light in the wavelength range of the treatment light and the visible light.

Further, the image collection unit 40 (see FIG. 1) includes a processor, such as, e.g., a GPU (Graphics Processing Unit) and an FPGA (Field-Programmable Gate Array) configured for image processing.

The image collection unit 40 is configured to receive the fluorescence signal detected by the fluorescence detection unit 33 and the signal of visible light including the treatment light detected by the treatment light detection unit 34 as electric signals. That is, the image collection unit 40 is configured such that the image data of the fluorescent image 81 (see FIG. 2) captured by the fluorescence detection unit 33 and the image data of the treatment light image 82 (see FIG. 3) captured by the treatment light detection unit 34 are input as electric signals. The image collection unit 40 is configured to collect the fluorescence signal and the signal of the visible light including the treatment light (data of the fluorescent image 81 and the treatment light image 82) based on the time series. The image collection unit 40 is configured to perform collecting or a stop of collecting the fluorescence signal and perform collecting or a stop of collecting the signal of the visible light, under the control of the device control unit 61, which will be described later. In the data of the fluorescence signal and the visible light signal collected by the image collection unit 40, the data before a predetermined time interval is updated at any time, or the data in a predetermined time period is updated at any time. The data of fluorescence signal and the data of the visible light are updated at any time in, for example, the storage unit 50, which will be described later.

The storage unit 50 (see FIG. 1) is configured to store (save) the fluorescence signal of the fluorescence detected by the light detection unit 30 (fluorescence detection unit 33). The storage unit 50 is configured to store (save) the signal of the visible light including the treatment light detected by the light detection unit 30 (treatment light detection unit 34). The storage unit 50 is configured to store (save) the fluorescence signal and the signal of the visible light including the treatment light (the data of the fluorescent image 81 and the data of the treatment light image 82), which are collected by the image collection unit 40. The storage unit 50 stores (saves) the fluorescence signal and the signal of the visible light including the treatment light collected based on a time series by the image collection unit 40 (the data of the fluorescent image 81 and the data of the treatment light image 82) together with a time stamp, such as, e.g., the date and time of imaging.

The storage unit 50 includes, for example, a nonvolatile memory, a hard disk drive (HDD: Hard Disk Drive), an SSD (Solid State Drive), and the like. The storage unit 50 may include a database connected by a network provided outside the treatment support device 20. With this configuration, the storage unit 50 is configured such that the fluorescence signal of the fluorescence detected by the light detection unit 30 (fluorescence detection unit 33) and the signal of the visible light including the treatment light detected by the light detection unit 30 (treatment light detection unit 34) can be stored for a long period of time. Further, in the storage unit 50, a program used for processing the control of the treatment support device 20 by the device control unit 61 to be described later. The storage unit 50 stores (stores) programs executed by the control unit 60 when collecting image data by the image collection unit 40.

The control unit 60 is provided with, as shown in FIG. 1, a device control unit 61 and a PC (Personal Computer) 62.

The device control unit 61 includes a control board (circuit board) on which a CPU (Central Processing Unit), a ROM (Read Only Memory), a RAM (Random Access Memory), and the like are mounted. The device control unit 61 is configured to control the entire treatment support device 20. The device control unit 61 is one example of the “first storage control unit” recited in claims. The device control unit 61 is configured to perform control at least a start of storing the fluorescence signal by the storage unit 50 when the PC 62 determines that the fluorescence signal detected by the light detection unit 30 has changed beyond at least a predetermined degree of change.

The PC 62 includes a CPU, a ROM, a RAM, and the like. The PC 62 is configured to analyze the fluorescence signal and the signal of visible light including the treatment light (the data of the fluorescent image 81 and the data of the treatment light image 82), which are collected by the image collection unit 40. The PC 62 is configured to analyze the information on the fluorescence signal strength for each time series in the fluorescent image 81. The PC 62 is an example of the “change determination unit” recited in claims. The PC 62 is configured to determine whether or not the fluorescence signal detected by the light detection unit 30 have changed beyond at least a predetermined degree of change. Further, the device control unit 61 and the PC 62 may be integrally formed.

Further, the operation units 71 and 72 (see FIG. 1) are user interfaces for operating the treatment support device 20. The operation unit 71, 72 includes, for example, a remote controller, a touch panel, a keyboard, a mouse, and the like. A touch panel as the operation unit 71 or the operation unit 72 may be provided on the display unit 80.

The operation unit 71 is configured to receive an operation relating to the control of the treatment support device 20 by the device control unit 61, such as, e.g., an operation for a start of detecting the fluorescence (collection of the fluorescence signal), an operation for a stop of detecting the fluorescence (collection of the fluorescence signal), and an operation for a start of detecting the visible light including the treatment light (collecting the signal of the visible light). The operation unit 72 is configured to receive an operation to the PC 62 that analyzes the fluorescence signal and the signal of the visible light including the treatment light (the data of the fluorescent image 81 and the treatment light image 82). The operation unit 72 is configured to receive an operation for setting a region of interest (ROI: Region Of Interest) for selectively acquiring the fluorescence signal, for example. Note that the operation for setting the region of interest for selectively acquiring the fluorescence signal may be configured to be received by the operation unit 71.

The display unit 80 (see FIG. 1) is configured by, for example, a liquid crystal display, or an organic EL (electroluminescent) display. The display unit 80 is connected to the control unit 60 (the device control unit 61 and the PC 62) by a video interface, such as, e.g., an HDMI (registered mark).

As shown in FIG. 2, the display unit 80 is configured to display a fluorescent image 81. The fluorescent image 81 is an image showing the distribution status of the fluorescence emitted by the fluorescent material of the medical agent 102. A user, such as, e.g., a physician, can confirm the integration of the medical agent 102 containing the fluorescent material bound to the cancer cells, from the distribution 81a of the fluorescence in the fluorescent image 81.

Further, as shown in FIG. 3, the display unit 80 is configured to display the treatment light image 82. In this embodiment, the treatment light image 82 is an image based on the treatment light and the visible light. In the treatment light image 82, in addition to the visible light, the treatment light leaked out of the cancer patient 101 from the insertion site of the treatment probe 12 and the treatment light transmitted through the body of the cancer patient 101 are reflected. The user, such as, e.g., a doctor, can confirm the position of the treatment probe 12 inserted into the cancer patient 101 and the treatment light irradiated from the treatment probe 12 by the treatment light image 82.

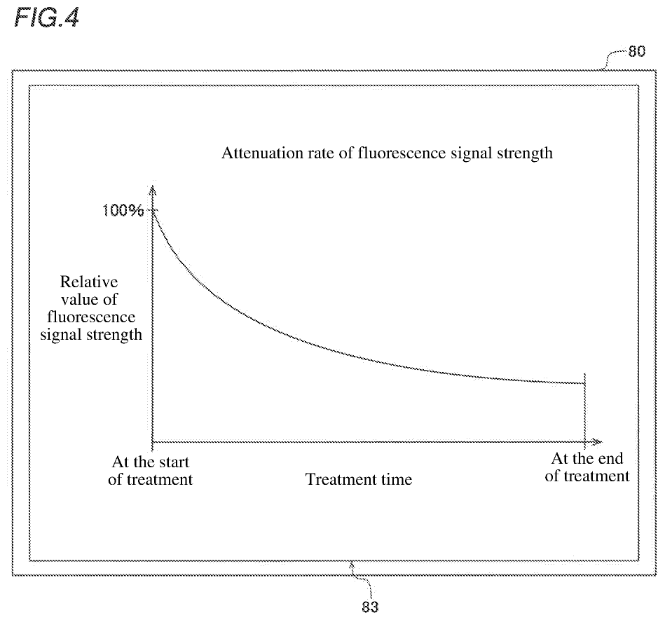

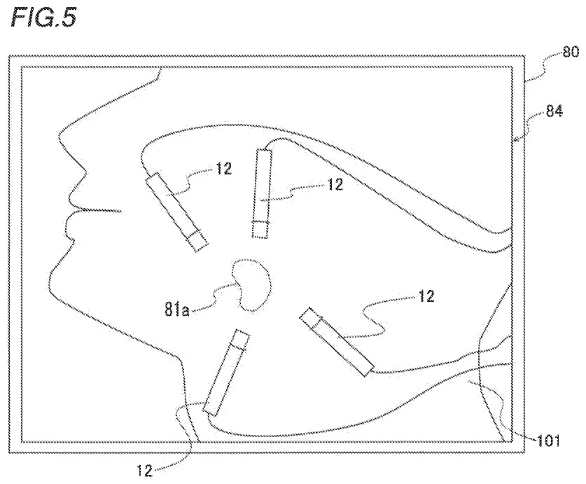

As shown in FIG. 4, the display unit 80 is configured to display the image 83 indicating the change in the fluorescence signal strength detected by the light detection unit 30 (the fluorescence detection unit 33). More specifically, the display unit 80 displays the image (the image 83 indicating the change in the fluorescence signal strength) showing the attenuation rate of the acquired fluorescence signal strength along the time series (treatment time) during the treatment with the fluorescence signal strength at the start of storing the fluorescence signal by the storage unit 50 (at the start of the treatment) being 100%. The display unit 80 starts displaying the image 83 indicating the change in the fluorescence signal strength with the start of storing (saving) the fluorescence signal by the storage unit 50 by the control of the control unit 60 (device control unit 61) to be described later. As a result, the user, such as, e.g., a doctor, can recognize the change in the acquired fluorescence signal strength in accordance with the time series.

The image 83 showing the change in the fluorescence signal strength displayed on the display unit 80 may be an image in which only the numerical value is displayed, or an image in which the graph and the numerical value are displayed at the same time, instead of the graph showing the change in the relative value of the fluorescence signal strength during the treatment (see FIG. 4). Further, the change in the fluorescence distributions 81a as shown in FIG. 2 may be displayed as the image 83 showing the change in the fluorescence signal strength. The display unit 80 stops displaying the image 83 indicating the change in the fluorescence signal strength with the stop of storing (saving) the fluorescence signal by the storage unit 50, under the control by the control unit 60 (device control unit 61) to be described later.

Further, as shown in FIG. 5, the display unit 80 may display a composite image 84 in which the fluorescent image 81 collected by the fluorescence detection unit 33 and the treatment light image 82 are superimposed. With this, the user, such as, e.g., a doctor, can simultaneously confirm the distribution 81a of the fluorescence displayed on the display unit 80, the position of the treatment probe 12, the position of the treatment light, and the position of treatment light. The composite image 84 is generated by superimposing the image data of the fluorescent image 81 collected by the image collection unit 40 and the image data of the treatment light image 82, by the image collection unit 40 or the control unit 60.

Further, in the display unit 80, the fluorescent image 81, the treatment light image 82 or the composite image 84, and the image 83 showing the change in the fluorescence signal strength may be displayed side by side at the same time. The display unit 80 may switch and display any one of the fluorescent image 81, the treatment light image 82, the composite image 84, and the image 83 indicating the change in the fluorescence signal strength, or may display any one of the images, or may all of the images at the same time, or may display any of the plurality of images in a superimposed manner.

### (Configuration for Control of Storage Unit by Control Unit and Control of Display Image by Control Unit)

The control unit 60 in the treatment support system 100 (the treatment support device 20) is configured such that the device control unit 61 controls at least the start of storing (saving) the fluorescence signal by the storage unit 50 based on that the fluorescence signal (the fluorescence signal strength) detected by the light detection unit 30 has changed beyond at least a predetermined degree of change.

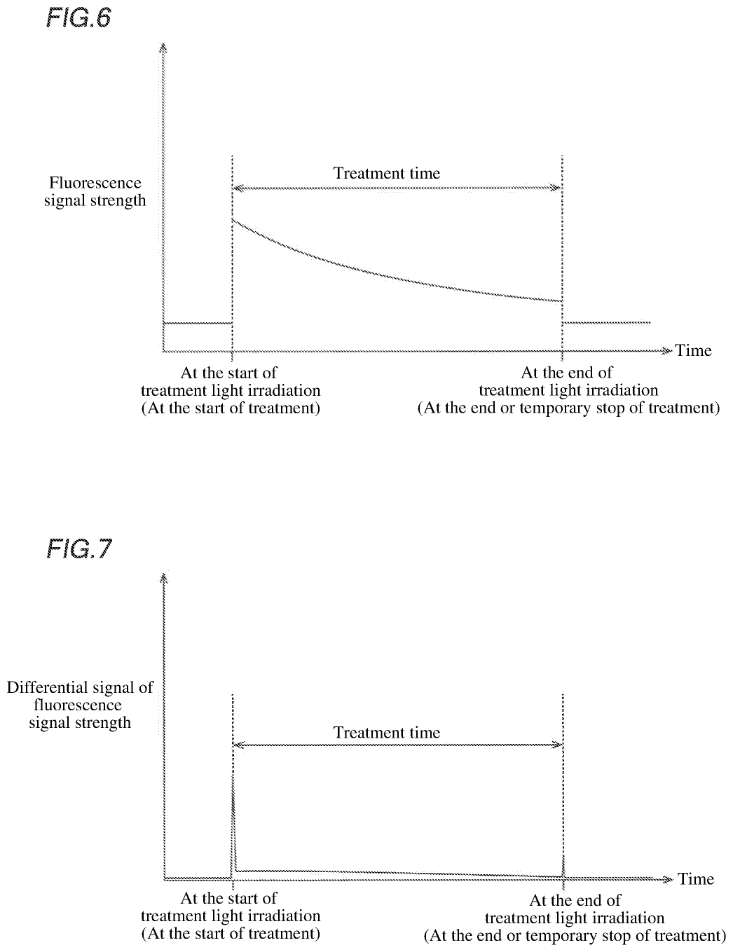

Here, when the irradiation of the treatment light from the irradiation unit 10 is started and the treatment by photoimmunotherapy is started, the fluorescent material contained in the medical agent 102 used in the treatment is excited, so that fluorescence is emitted from the fluorescent material contained in the medical agent 102. Therefore, as shown in FIG. 6, in the fluorescence signal detected by the light detection unit 30 (the fluorescence detection unit 33), the rise (rapid rise) of the fluorescence signal strength occurs at the time of starting the treatment.

Further, when the irradiation of the treatment light from the irradiation unit 10 is stopped and the treatment is terminated, the fluorescent material contained in the medical agent 102 used in the treatment is not excited, so that fluorescence cannot be emitted from the fluorescent material contained in the medical agent 102. Therefore, as shown in FIG. 6, in the fluorescence signal detected by the light detection unit 30 (the fluorescence detection unit 33), the fall of the fluorescence signal strength (abrupt fall) occurs at the time of ending (at the time of pausing) the treatment. Therefore, as shown in FIG. 7, a spike-like signal change occurs in the differential (difference) signal of the fluorescence signal strength before and after starting the treatment and the differential (difference) signal of the fluorescence signal strength before and after ending the treatment.

At predetermined intervals, the control unit 60 (the PC 62) acquires the fluorescence signal (fluorescence signal strength) collected by the image collection unit 40 and monitors (observes) the change in the fluorescence signal (the fluorescence signal strength). The control unit 60 (the PC 62) is configured to compare the current fluorescence signal collected by the image collection unit 40 with the fluorescence signal collected by the image collection unit 40, which are detected by the light detection unit 30 (the fluorescence detection unit 33). With this, the control unit 60 (the PC 62) is configured to acquire the change in the fluorescence signal (the fluorescence signal strength) due to the start of emitting the treatment light (treatment start) and the stop of emitting the treatment light (treatment end) as described above.

The predetermined time interval in which the control unit 60 (PC 62) monitors (observes) the fluorescence signal (the fluorescence signal strength) collected by the image collection unit 40 is, for example, 100 ms. In a case where the irradiation unit 10 emits pulsed treatment light, it is configured such that the current fluorescence signal collected by the image collection unit 40 is compared with the fluorescence signal before the predetermined time interval collected by the image collection unit 40 at the timing (time interval) corresponding to (synchronous with) the timing of emitting the treatment light by the irradiation unit 10.

The control unit 60 (the PC 62) may monitor (observe) either the average of the fluorescence signals (fluorescence signal strength) in the entire region (the entire fluorescent image 81) detected by the fluorescence detection unit 33 or the fluorescence signal in the pixel region within the set region of interest. The fluorescence signal (fluorescence signal strength) that the control unit 60 monitors (observes) may be the fluorescence signal detected in one region, the the fluorescence signal detected in a plurality of regions, the fluorescence signal detected by one pixel of the image sensor, or the fluorescence signal detected by a plurality of pixels of the image sensor.

The control unit 60 is configured such that the device control unit 61 performs control of a start of storing (saving) a fluorescence signal by the storage unit 50 based on that the fluorescence signal strength detected by the light detection unit 30 (the fluorescence detection unit 33) has increased beyond a predetermined degree of change. In the first embodiment, the control unit 60 is configured to perform control of a start of storing the fluorescence signal by the storage unit 50 based on that the rate of change in the fluorescence signal strength detected by the light detection unit 30 has exceeded a threshold.

In the first embodiment, the control unit 60 is configured to perform control of a start of storing (saving) the fluorescent image signal by the storage unit 50 when the difference between the gradation of the current fluorescence and the gradation of the fluorescent image before the predetermined time interval has exceeded the threshold (start threshold) for starting the recording of the fluorescence signal by the storage unit 50. Note that as described above, the fluorescent image 81 is a pixel based on the fluorescence signal (the fluorescence signal strength) detected by the light detection unit 30 (the fluorescence detection unit 33).

In particular, the control unit 60 is configured to perform control of a start of storing (saving) the fluorescence signal by the storage unit 50 when the gradation of the current fluorescent image has increased by the start threshold or more relative to the gradation of the fluorescent image before the predetermined timer intervals. For example, in a case where the image collection unit 40 or the control unit 60 (the PC 62) processes the fluorescence signal strength from 0 to 255 gradations (256 gradations) to be displayed on the display unit 80 as an image (fluorescent image 81), when the difference between the current gradation and the gradation before the predetermined time interval (e.g., before 100 ms) has increased by 30 or more, the PC 62 determines that the fluorescence signal strength has increased (changed) beyond the predetermined degree of change, and sends a command of a start of storing to the device control unit 61. Then, the device control unit 61 is configured to receive the command for a start of recording from the PC 62 and perform control of a start of storing the fluorescence signal by the storage unit 50. The value of the threshold (start threshold) for starting the recording of the fluorescence signal by the storage unit 50 may be changed based on the illumination strength of the treatment light, or may be changed by the user's operation.

Note that the control unit 60 (the PC 62) may be configured to perform control of a start of storing the fluorescence signal by the storage unit 50 when the difference between the value (numerical value) of the current fluorescence signal strength and the value (numerical value) of fluorescence signal strength before the predetermined time interval has increased beyond the threshold (start threshold) for starting the recording of the fluorescence signal by the storage unit 50 by the storage unit 50.

Furthermore, the control unit 60 is configured such that based on that the fluorescence signal strength detected by the light detection unit 30 has increased beyond the predetermined degree of change, the device control unit 61 performs control of a start of storing (saving) the fluorescence signal strength detected by the storage unit 50 and perform control of a start of displaying the image 83 indicating the change in the fluorescence signal strength detected by the light detection unit 30 on the display unit 80.

Specifically, the control unit 60 is configured such that based on the fluorescence signal strength detected by the light detection unit 30 (the fluorescence detection unit 33) has increased beyond the above-described start threshold, the device control unit 61 performs control of the start of displaying the image 83 indicating the change in the fluorescence signal strength. Note that the control of the start of displaying the image 83 indicating the change in the fluorescence signal strength and the control of the start of storing the fluorescence signal by the storage unit 50 may be performed with a time difference.

Further, the control unit 60 is configured such that based on the fluorescence signal strength detected by the light detection unit 30 has decreased beyond the predetermined degree of change, the device control unit 61 performs control of a stop of storing (saving) the fluorescence signal by the storage unit 50.

In the first embodiment, the control unit 60 is configured, when the difference between the gradation of the current fluorescent image and the gradation of the fluorescent image before the predetermined time interval has exceeded the threshold (stop threshold) for stopping the recording by the storage unit 50, to perform control of a stop of storing (saving) the fluorescence signal by the storage unit 50. More specifically, when the gradation of the current fluorescent image has decreased beyond the stop threshold with respect to the gradation of the fluorescent image before the predetermined time interval, the PC 62 determines that the fluorescence signal strength detected by the light detection unit 30 has decreased beyond the predetermined degree of change and the command for stopping the storing to the device control unit 61. Then, the device control unit 61 is configured to receive the command for stopping the recording from the PC 62 and control the stop of storing (saving) the fluorescence signal by the storage unit 50.

Here, as shown in FIG. 6, in photoimmunotherapy, the fluorescence (fluorescence signal strength) emitted by the fluorescent material of the medical agent 102 decreases as the treatment by photoimmunotherapy progresses (as the irradiation time of the treatment light becomes longer). Therefore, as shown in FIG. 7, the differential (difference) signal of the fluorescence signal strength before and after the end of the end of the treatment (the pause of the treatment) is smaller than the differential (difference) signal of the fluorescence signal strength before and after the start of the treatment. Therefore, the threshold (stop threshold) for performing control of a stop of storing the fluorescence signal by the storage unit 50 is smaller than the threshold (start threshold) for starting the recording by the storage unit 50. The value of the threshold (stop threshold) for performing control of the stop of storing the fluorescence signal by the storage unit 50 may be changed based on the illumination strength of the treatment light or may be changed by the user's operation.

Note that the control unit 60 may be configured to perform control of a stop of storing (saving) the fluorescence signal by the storage unit 50 when the difference between the value (numerical value) of the current fluorescence signal strength and the value (numerical value) of the fluorescence signal strength before the predetermined time interval has decreased beyond the threshold (stop threshold) for performing the stop of recording by the storage unit 50.

Furthermore, the control unit 60 is configured such that, based on that the fluorescence signal strength detected by the light detection unit 30 (fluorescence detection unit 33) has decreased beyond the predetermined degree of change, the device control unit 61 performs control of the stop of storing (saving) the fluorescence signal by the storage unit 50 and performs control of the stop of displaying the image 83 indicating the change in the fluorescence signal strength detected by the light detection unit 30 on the display unit 80.

Specifically, the control unit 60 is configured such that the device control unit 61 performs a control of a stop of displaying the image 83 indicating the change in the fluorescence signal strength displayed on the display unit 80 based on that the fluorescence signal strength detected by the light detection unit 30 (the fluorescence detection unit 33) has changed beyond the start threshold, based on that the fluorescence signal strength detected by the light detection unit 30 has decreased beyond the stop threshold. Note that the control of a stop of displaying the image 83 indicating the change in the fluorescence signal strength and the control of a stop of storing the fluorescence signal by the storage unit 50 may be carried out with a time lag.

Note that the control of a start of storing (saving) the fluorescence signal by the storage unit 50, the control of a stop of storing (saving) the fluorescence signal by the storage unit 50, the control of a start of displaying the image 83 indicating the change in the fluorescence signal strength, and the control of a stop of displaying the image 83 indicating the change in the fluorescence signal strength may be performed by the PC 62 of the control unit 60 via the device control unit 61 with respect to the image collection unit 40, the storage unit 50, and the display unit 80. Alternatively, the PC 62 of the control unit 60 may directly perform without intervening the device control unit 61.

### Effects of First Embodiment

In this embodiment, the following effects can be obtained.

In the first embodiment, the control unit 60 of the treatment support system 100 (the treatment support device 20) is configured such that based on that the fluorescence signal detected by the light detection unit 30 has changed beyond at least the predetermined degree of change, the device control unit 61 (the first storage control unit) performs control of at least a start of storing (saving) the fluorescence signal by the storage unit 50.

Here, when the irradiation of the treatment light is started and the treatment is started, the fluorescent material contained in the medical agent 102 used in the treatment is excited, and the fluorescence is emitted from the fluorescent material contained in the medical agent 102. Therefore, when the irradiation of the treatment light is started and the treatment is started, the strength of the fluorescence signal changes abruptly. As a result, the control unit 60 (the device control unit 61) can acquire the timing of the treatment start based on the fact that the fluorescence signal detected by the light detection unit 30 has changed beyond at least the predetermined degree of change. Therefore, the control unit 60 can perform the control of a start of storing (saving) the fluorescence signal storage by the storage unit 50 at the timing of the treatment start without any time difference. As a result, it is possible to start storing the fluorescence signal without any time difference at the timing of the start of the treatment.

Further, in the configuration in which the irradiation unit 10 and the treatment support device 20 (the light detection unit 30) are separately provided as in the treatment support system 100, the control unit 60 can acquire the timing of the treatment start based on that the fluorescence signal detected by the light detection unit 30 has changed beyond at least the predetermined degree of change. As a result, since the storing of the fluorescence signal by the storage unit 50 can be started at the timing of the treatment start in synchronism with the start (treatment start) of the irradiation of the treatment light by the irradiation unit 10, the storing of the fluorescence signal can be started without any time difference at the timing of the treatment start by simply performing the operation of the start of irradiating the treatment light by the irradiation unit 10, even in the configuration in which the irradiation unit 10 and the treatment support device 20 (the light detection unit 30) are separately provided independently.

Further, in the treatment support system 100 according to the first embodiment described above, by configuring as described below, it is possible to obtain further effects as described below.

Further, in the treatment support system 100 of the first embodiment, as described above, the control unit 60 is configured such that based on that the fluorescence signal strength detected by the light detection unit 30 has changed beyond at least the predetermined degree of change, the device control unit 61 (the first storage control unit) performs control of at least a start of storing the fluorescence signal by the storage unit 50. As a result, the control unit 60 (the device control unit 61) can acquire the timing of the treatment start based on the fact that the fluorescence signal strength detected by the light detection unit 30 has changed beyond at least the predetermined degree of change, so that the start of storing the fluorescence signal by the storage unit 50 can be easily performed at the timing of the treatment start without a time lag. As a result, at the timing of the treatment start, the start of storing the fluorescence signal can be easily performed without a time lag.

Further, in the treatment support system 100 of the first embodiment, as described above, the control unit 60 is configured such that based on the fact that the rate of change in the fluorescence signal strength detected by the light detection unit 30 has exceeded the threshold, the device control unit 61 (the first storage control unit) performs control of at least a start of storing the fluorescence signal by the storage unit 50. As a result, since the control unit 60 (the device control unit 61) can acquire the timing of the treatment start based on that the rate of change in the fluorescence signal strength detected by the light detection unit 30 has exceeded the threshold, the storage unit 50 can start storing the fluorescence signal at the timing of the treatment start more easily without a time lag. As a result, the storing of the fluorescence signal can be started more easily without a time lag at the timing of the treatment start.

Further, in the treatment support system 100 of the first embodiment, as described above, the control unit 60 is configured such that based on that the fluorescence signal strength detected by the light detection unit 30 has increased beyond the predetermined degree of change in the fluorescence signal, the device control unit 61 (the first storage control unit) performs control of a start of storing (saving) the fluorescence signal by the storage unit 50. Here, when the irradiation of the treatment light is started and the treatment is started, the fluorescent material contained in the medical agent 102 used in the treatment is excited, so that fluorescence is emitted from the fluorescent material contained in the medical agent 102.

Therefore, when the irradiation of the treatment light is started and the treatment is started, the strength of the fluorescence signal increases rapidly. As a result, the control unit 60 (the device control unit 61) can acquire the timing of the start of the treatment based on the fact that the fluorescence signal strength detected by the light detection unit 30 has increased beyond the predetermined degree of change, so that the control of the start of storing the fluorescence signal by the storage unit 50 can be performed at the timing of the treatment start more easily without a time lag. As a result, at the timing of the treatment start, the start of storing the fluorescence signal can be performed more easily without a time lag.

Further, in the treatment support system 100 of the first embodiment, as described above, the control unit 60 is configured such that based on the fact that the fluorescence signal strength detected by the light detection unit 30 has increased beyond the predetermined degree of change, the device control unit 61 (the first storage control unit) performs control of a start of storing the fluorescence signal by the storage unit 50 and also performs control of a start of displaying the image 83 indicating the change in the fluorescence signal strength detected by the light detection unit 30 on the display unit 80.

With this, since the control of a start of storing the fluorescence signal by the storage unit 50 and the control of a start of displaying the image 83 indicating the change in the fluorescence signal strength are performed, the user can easily recognize that the storing of the fluorescence signal by storage unit 50 has started by the display of the image 83 indicating the change in the fluorescence signal strength.

Further, in the treatment support system 100 of the first embodiment, as described above, the control unit 60 is configured such that the device control unit 61 (the first storage control unit) performs control of a stop of storing the fluorescence signal by the storage unit 50 based on the fact that the fluorescence signal strength detected by the light detection unit 30 has decreased beyond the predetermined degree of change.

Here, when the irradiation of the treatment light is stopped and the treatment is completed, the fluorescent material contained in the medical agent 102 used for the treatment is not excited, so that fluorescence cannot be emitted from the fluorescent material contained in the medical agent 102. Therefore, when the irradiation of the treatment light is stopped and the treatment is finished, the strength of the fluorescence signal decreases suddenly. As a result, the control unit 60 (the device control unit 61) can acquire the timing of the end of the treatment based on the fact that the fluorescence signal strength detected by the light detection unit 30 has decreased beyond the predetermined degree of change, so that the storing of the fluorescence signal by the storage unit 50 can be stopped at the timing of the end of the treatment without a time difference. As a result, the storing of the fluorescence signal can be stopped without a time difference at the timing of the end of the treatment.

Further, in the treatment support system 100 of the first embodiment, as described above, the control unit 60 is configured such that based on the fact that the fluorescence signal strength detected by the light detection unit 30 has decreased beyond the predetermined degree of change, the device control unit 61 (the first storage control unit) performs control of a stop of storing (saving) the fluorescence signal by the storage unit 50 and also performs control of a stop of displaying the image 83 indicating the change in the fluorescence signal strength detected by the light detection unit 30 in the display unit 80. With this, since the control of a stop of storing the fluorescence signal by the storage unit 50 and the control of a stop of displaying the image 83 indicating the change in the fluorescence signal strength are performed, the user can easily recognize that the storing of the fluorescence signal by the storage unit 50 has stopped by the stop of the display of the image 83 indicating the change in the fluorescence signal strength.

### Second Embodiment

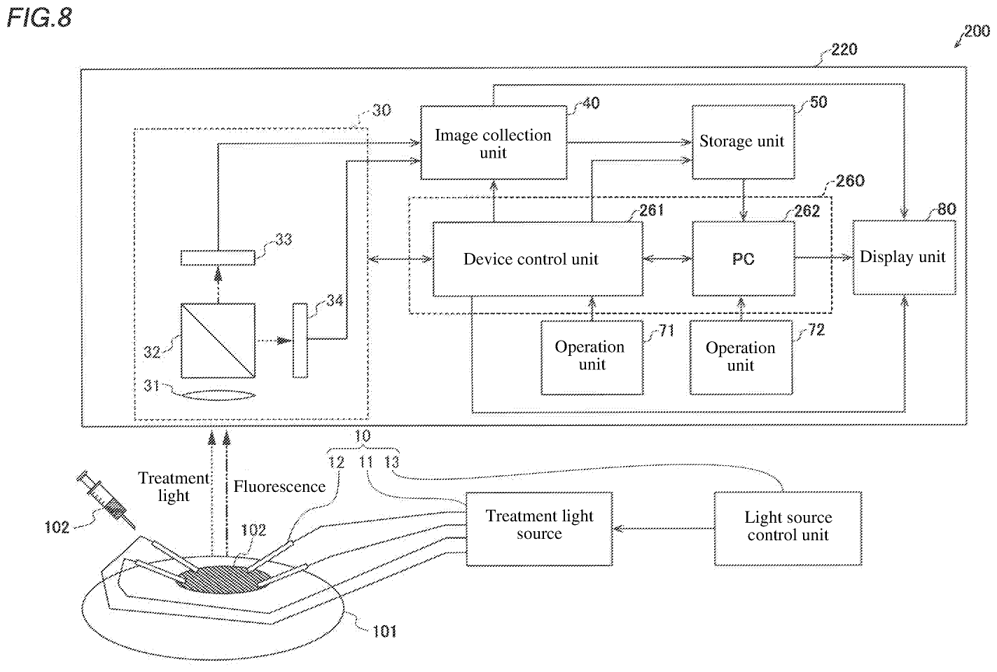

Referring to FIG. 8, a configuration of a treatment support system 200 (treatment support device 220) according to a second embodiment will be described. Note that in the figures, the same component as that of the first embodiment is denoted by the same reference symbol.

In the second embodiment, unlike the first embodiment in which the control of a start of storing the fluorescence signal by the storage unit 50 is performed based on the fluorescence signal strength detected by the light detection unit 30 has changed beyond at least the predetermined degree of change, it is configured to perform control of a start of storing the light detection unit signal by the storage unit 50 based on that the treatment light signal is detected by the treatment light detection unit 34.

As shown in FIG. 8, the light detection unit 30 of the treatment support system 200 (the treatment support device 220) is provided with a fluorescence detection unit 33 and the treatment light detection unit 34, similarly to the treatment support system 100 of the first embodiment. The fluorescence detection unit 33 is configured to detect the fluorescence emitted by the fluorescent material of the medical agent 102 excited by the irradiation of the treatment light. The treatment light detection unit 34 is configured to detect visible light including the treatment light.

The control unit 260 is configured to perform control of at least a start of storing (saving) the fluorescence signal by the storage unit 50 based on that the treatment light detection unit 34 has detected the treatment light signal. The control unit 260 includes a device control unit 261 and a PC 262 as shown in FIG. 8. The PC 262 is configured to determine whether or not the light detection unit 30 has detected the treatment light signal. The device control unit 261 is configured to perform control of a start of storing the fluorescence signal by the storage unit 50 when the PC 262 determines that the light detection unit 30 has detected the treatment light signal. Note that the device control unit 261 is an example of the “second storage control unit” recited in claims, and the PC 262 is an example of the “treatment light determination unit” recited in claims.

Specifically, the control unit 260 is configured such that, when a pixel (region of a pixel) having a gradation of a color corresponding to the light in the wavelength range of the treatment light equal to or larger than a predetermined value is detected in the treatment light image 82 (see FIG. 3), the PC 262 determines that the signal of the treatment light is detected and the device control unit 261 performs control of at least a start of storing the fluorescence signal by the storage unit 50.

For example, in a case where the wavelength range of the treatment light is about 600 nm to 800 nm, the treatment light is in a region of from red light to near-infrared light. Therefore, the control unit 260 (the PC 262) determines that the treatment light signal has been detected when a pixel (region of a pixel) whose gradation of R is equal to or greater than a predetermined value has been detected in the treatment light image 82 among R (Red), G (Green), and B (Blue). In other words, when the hue of the light detected by the treatment light detection unit 34 has changed to the same hue as the treatment light, the control unit 260 (the PC 262) determines that the signal of the treatment light has been detected.

Note that when detecting the signal of the treatment light, the control unit 260 (the PC 262) may determine whether or not the signal of the treatment light has been detected based on the value of any gradation of R, G, and B in the treatment light image 82, or may determine whether or not the signal of the treatment light has been detected based on the detection of the region of the pixel whose value of gradation of R, G, and B is equal to or greater than the threshold set for each of R, G, and B.

Further, the treatment support system 200 may be configured such that the device control unit 261 performs control of a stop of storing (saving) the fluorescence signal by the storage unit 50 based on the fact that the treatment light detection unit 34 no longer detects the signal of the treatment light. That is, in the control unit 260, when a pixel (region of a pixel) whose gradation of the color corresponding to the light in the wavelength range of the treatment light is equal to or more than a predetermined value is not detected in the treatment light image 82, the PC 262 may determine that the signal of the treatment light is not detected, and the device control unit 261 performs the control of a stop of storing the fluorescence signal by the storage unit 50.

Further, in the treatment support system 200, the control unit 260 (the device control unit 261) may be configured to perform control of a start of storing the fluorescence signal by the storage unit 50 and perform control of displaying the image 83 indicating the change in the fluorescence signal strength detected by the light detection unit 30 on the display unit 80. Further, in the treatment support system 200, the control unit 260 (the device control unit 261) may be configured to perform control of a stop of storing the fluorescence signal by the storage unit 50 and perform control of a stop of displaying the image 83 indicating the change in the fluorescence signal strength detected by the light detection unit 30 on the display unit 80.

The rest of the configuration of the second embodiment is the same as that of the first embodiment.

### Effects of Second Embodiment

In this second embodiment, the following effects can be acquired.

In the second embodiment, the control unit 260 of the treatment support system 200 (the treatment support device 220) is configured such that the device control unit 261 (the second storage control unit) performs control of at least a start of storing the fluorescence signal by the storage unit 50 based on that the light detection unit 30 has detected the signal of the treatment light.

Here, since the treatment is started by emitting the treatment light by the irradiation unit 10, the signal of the treatment light is detected when the treatment is started. As a result, the control unit 260 (the device control unit 261) can acquire the timing of starting the treatment based on the fact that the light detection unit 30 has detected the signal treatment light. Therefore, the storage unit 50 can perform control of a start of storing the fluorescence signal at the timing of the start of the treatment without a time difference. As a result, it is possible to provide a treatment support system 200 capable of performing a start of storing the fluorescence signal without a time difference at the timing of the start of the treatment.

Also even in a configuration in which the irradiation unit 10 and the treatment support device 220 (the light detection unit 30) are separately provided as in the case of the treatment support system 200, the control unit 260 can acquire the timing of a start of the treatment based on the fact that the light detection unit 30 has detected the signal of the treatment light. As a result, the storing of the fluorescence signal by the storage unit 50 can be started at the timing of the start of the treatment in synchronization with the start of the irradiation of the treatment light by the irradiation unit 10 (the start of the treatment) by simply performing the operation of the start of emitting the treatment light by the irradiation unit 10. Therefore, even in the configuration in which the irradiation unit 10 and the treatment support device 220 (the light detection unit 30) are separately provided independently, the start of storing the fluorescence signal can be performed without a delay at the timing of the start of the treatment.

The other effects of the second embodiment are the same as those of the above-described first embodiment.

### Third Embodiment

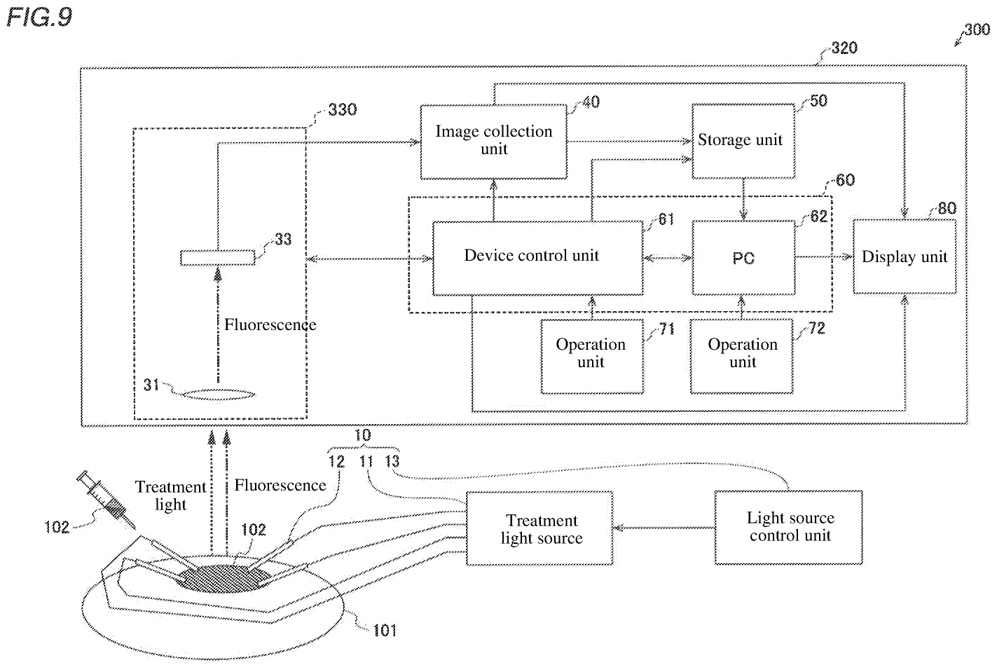

Referring to FIG. 9, a configuration of a treatment support system 300 (a treatment support device 320) according to a third embodiment will be described. In the figures, the same component as that of the first and second embodiments is denoted by the same reference numeral.

Unlike the first and second embodiments in which the light detection unit 30 includes the fluorescence detection unit 33 and and the treatment light detection unit 34, the light detection unit 330 of the treatment support system 300 (the treatment support device 320) according to the third embodiment is configured to include only a fluorescence detection unit 33, as shown in FIG. 9. As a result, the device configuration of the treatment support system 300 (the light detection unit 30) can be simplified.

The other structures and effects of the third embodiment are the same as those of the first and second embodiment.

### Modified Embodiment

It should be understood that the embodiments disclosed here are examples in all respects and are not restrictive. The scope of the present invention is shown by the scope of the claims rather than the descriptions of the embodiments described above, and includes all changes (modifications) within the meaning of equivalent and the scope of claims.

For example, in the first to third embodiments described above, examples are shown. In one example, the control unit 60 is configured to perform control of a start of storing the fluorescence signal by the storage unit 50 based on that the fluorescence signal detected by the light detection unit 30 (the fluorescence detection unit 33) has changed beyond at least a predetermined degree of change. In another example, the control unit 260 is configured to perform control of a start of storing the fluorescence signal by the storage unit 50 based on the detection of the treatment light signal by the light detection unit 30 (the treatment light detection unit 34). However, the present invention is not limited to the above-described examples.

In the present invention, the control unit may be configured to perform control of a start of storing the fluorescence signal based on both the facts that the fluorescence signal detected by the light detection unit has changed beyond at least a predetermined degree of change and that the light detection unit has detected the signal of the treatment light. That is, the control unit may be configured to perform control of a start of storing the fluorescence signal based on both the result of the determination of the change determination unit and the result of the determination of the treatment light determination unit.

Further, in the first and third embodiments, an example is shown in which the control unit 60 is configured such that, based on the rate of change in the fluorescence signal strength detected by the light detection unit 30 has exceeded the threshold (start threshold), the device control unit 61 (the first storage control unit) performs control of a start of storing fluorescence signal by the storage unit 50, but the present invention is not limited thereto.

In the present invention, the control unit may be configured such that the first storage control unit performs control of a start of storing the fluorescence signal by the storage unit based on the fact that the current fluorescence signal strength detected by the light detection unit has exceeded the threshold. That is, the control unit may be configured such that the first storage control unit performs control of a start of recording the fluorescence signal based on the absolute value of the signal strength of the fluorescence light by the storage unit.

Further, in the first and third embodiments, an example is shown in which the control unit 60 is configured such that, based on the fact that the fluorescence signal strength detected by the light detection unit 30 has increased beyond the predetermined degree of change, the device control unit 61 (the first storage control unit) performs control of a start of storing the fluorescence signal by the storage unit 50 and the displaying of the image 83 indicating the change in the fluorescence signal strength detected by the light detection unit 30 on the display unit 80, but the present invention is not limited thereto.

In the present invention, the control unit may be configured to perform only the control of a start of storing the fluorescence signal by the storage unit without starting the displaying of the image indicating the change in the fluorescence signal strength based on the fact that the fluorescence signal strength detected by the light detection unit has increased beyond the predetermined degree of change.

Further, in the first and third embodiments, an example is shown in which the control unit 60 is configured such that, based on the fluorescence signal detected by the light detection unit 30 (fluorescence detection unit 33) has changed beyond at least a predetermined degree of change, the device control unit 61 (first storage control unit) performs the control of the start and the stop of storing the fluorescence signal by the storage unit 50, but the present invention is not limited thereto.

In the present invention, the control unit may be configured to perform only control of a start of storing the fluorescence signal by the storage unit, without performing the control of a stop of storing the fluorescence signal by the storage unit.

Further, in the first and third embodiments described above, an example is shown in which the control unit 60 is configured such that based on the fact that the fluorescence light signal strength detected by the light detection unit 30 has decreased beyond the predetermined degree of change, the device control unit 61 (the first storage control unit) performs control of a stop of storing the fluorescence signal by the storage unit 50 and performs control of a stop of displaying the image 83 indicating the change in the fluorescence signal strength detected by the light detection unit 30 on the display unit 80. However, the present invention is not limited thereto.

In the present invention, the control unit may be configured to perform only the control of a stop of storing the fluorescence signal by the storage unit without stopping the display of the image indicating the change in the fluorescence signal strength based on the fact that the fluorescence signal strength detected by light detection unit has decreased beyond the predetermined degree of change.

In the first and third embodiments, an example is shown in which the control unit 60 includes the device control unit 61 (the first storage control unit) and the PC 62 (the change determination unit), but the present invention is not limited thereto. In the present invention, the control unit composed of a CPU or the like as hardware may include a first storage control unit and a change determination unit as functional blocks of software (programs).

In the second embodiment, an example is shown in which the control unit 260 includes the device control unit 261 (the second storage control unit) and the PC 262 (the treatment light determination unit), but the present invention is not limited thereto. In the present invention, the control unit composed of a CPU or the like as hardware may include a second storage control unit and a treatment light determination unit as functional blocks of software (programs). Further, in the present invention, the control unit composed of a CPU or the like as hardware may include a first storage control unit, a change determination unit, a second storage control unit, and a treatment light determination unit.

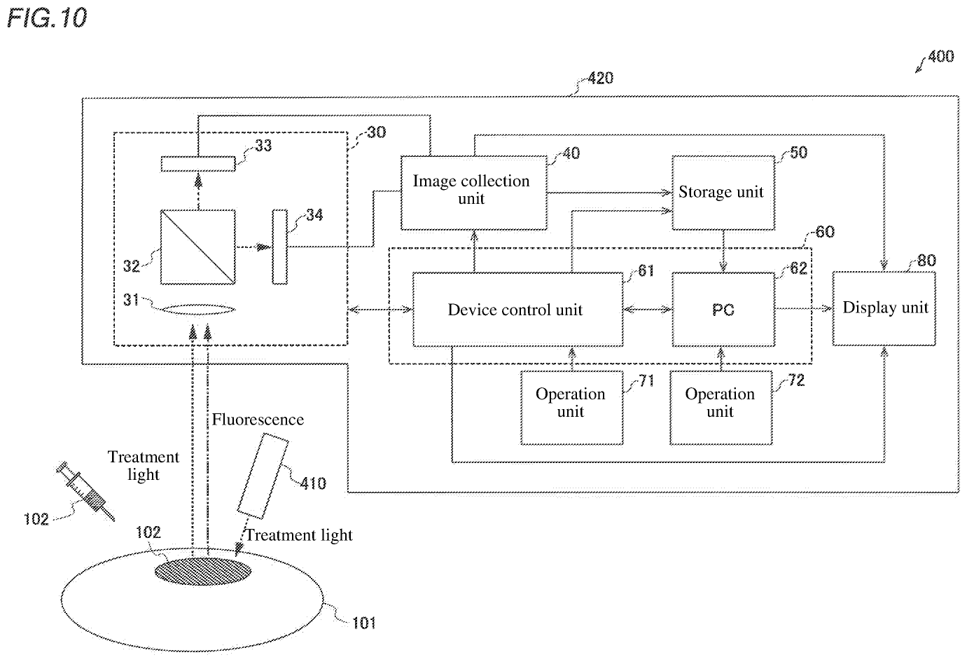

Further, in the first to third embodiments described above, an example is shown in which the irradiation unit 10 (treatment probe 12) emits the treatment light in the body of the cancer patient 101, but the present invention is not limited thereto. In the present invention, as in the treatment support system 400 (treatment support device 420) according to the first modification shown in FIG. 10, an irradiation unit 410 from outside the body of the cancer patient 101 may be configured to perform treatment light irradiation.

Note that the irradiation unit 410 includes a light source, such as, e.g., a semiconductor laser (LD: Laser Diode) and a light emitting diode (LED: Light Emitting Diode). Further, the irradiation unit 410 also includes a light control unit for controlling the turning on and off of the light source, such as, e.g., a semi-conductor laser and a light-emitting diode (i.e., the start and the stop of irradiation of the treatment light).

### [Aspects]

It will be understood by those skilled in the art that the above-described exemplary embodiments are concrete examples of the following aspects.

### (Item 1)

A treatment support system comprising:

an irradiation unit configured, in treatment of killing a cancer cell by irradiating a medical agent containing a fluorescent material that has been administered into a body of a subject with treatment light in a predetermined wavelength range, to irradiate the medical agent with the treatment light;

a light detection unit configured to detect, out of the treatment light and fluorescence emitted by the fluorescent material of the medical agent excited by irradiation of the treatment light, at least the fluorescence emitted by the fluorescent material of the medical agent excited by the irradiation of the treatment light;

a storage unit configured to store a fluorescence signal of the fluorescence detected by the light detection unit; and

a control unit configured to perform control of at least a start of storing the fluorescent signal by the storage unit,

wherein the control unit is configured to

include a change determination unit for determining whether or not the fluorescence signal detected by the light detection unit has changed beyond at least a predetermined degree of change and a first storage control unit for performing control of at least the start of storing the fluorescence signal by the storage unit when the change determination unit determines that the fluorescence signal detected by the light detection unit has changed beyond at least the predetermined degree of change, and

perform control of at least the start of storing the fluorescence signal by the storage unit based on that the fluorescence signal detected by the light detection unit has changed beyond at least the predetermined degree of change, or

wherein the control unit is configured to

include a treatment light determination unit for determining whether or not the light detection unit has detected a signal of the treatment light and a second storage control unit for performing control of the start of storing the fluorescence signal by the storage unit when the treatment light determination unit determines that the light detection unit has detected the signal of the treatment light, and

perform control of at least the start of storing the fluorescence signal by the storage unit based on that the light detection unit has detected the signal of the treatment light.

### (Item 2)

The treatment support system as recited in the above-described Item 1,

wherein the control unit is configured such that the first storage control unit performs control of at least the start of storing the fluorescence signal by the storage unit based on that fluorescence signal strength detected by the light detection unit has changed beyond at least the predetermined degree of change.

### (Item 3)

The treatment support system as recited in the above-described Item 2,

wherein the control unit is configured such that the first storage performs control of at least the start of storing the fluorescence signal by the storage unit based on that a rate of change of the fluorescence signal strength detected by the light detection unit has exceeded a thoreshold.

### (Item 4)

The treatment support system as recited in the above-described Item 2 or 3,

wherein the control unit is configured such that the first storage control unit performs control of the start of storing the fluorescence signal by the storage unit based on that the fluorescence signal strength detected by the light detection unit has increased beyond the predetermined degree of change.

### (Item 5)

The treatment support system as recited in the above-described Item 4, further comprising:

a display unit configured to display an image indicating a change in the fluorescence signal strength detected by the light detection unit,

wherein the control unit is configured such that, based on that the fluorescence signal strength detected by the light detection unit has increased beyond the predetermined degree of change, the first storage control unit performs control of the start of storing the fluorescence signal by the storage unit and performs control for a start of displaying the image indicating the change in the fluorescence signal strength detected by the light detection unit on the display unit.

### (Item 6)

The treatment support system as recited in the above-described Item 4 or 5,

wherein the control unit is configured such that the first storage control unit performs control of a stop of storing the fluorescence signal by the storage unit based on that the fluorescence signal strength detected by the light detection unit has decreased beyond a predetermined degree of change.

### (Item 7)

The treatment support system as recited in the above-described Item 5,

wherein the control unit is configured such that, based on that the fluorescence signal strength detected by the light detection unit has decreased beyond the predetermined degree of change, the first storage control unit performs control of the stop of storing the fluorescence signal by the storage unit and performs control of the stop of displaying the image indicating the change in the fluorescence signal strength detected by the light detection unit on the display unit.

### (Item 8)

The treatment support system as recited in any one of the above-described Items 1 to 7,

wherein the light detection unit includes a treatment light detection unit for detecting the treatment light and a fluorescence detection unit for detecting the fluorescence emitted by the fluorescent material of the medical agent excited by the irradiation of the treatment light, and

wherein the control unit is configured such that the first storage control unit performs control of at least the start of storing the fluorescence signal by the storage unit based on that the fluorescence signal detected by the fluorescence detection unit has changed beyond at least the predetermined degree of change, or the second storage control unit performs control of at least the start of storing the fluorescence signal by the storage unit based on that the treatment light detection unit has detected the signal of the treatment light.

### (Item 9)

A treatment support device comprising:

a light detection unit configured, in treatment of killing a cancer cell by irradiating a medical agent containing a fluorescent material that has been administered into a body of a subject with treatment light in a predetermined wavelength range, to detect, out of the treatment light and the fluorescence emitted by the fluorescent material of the medical agent excited by irradiation of the treatment light, at least the fluorescence emitted by the fluorescent material of the medical agent excited by the irradiation of the treatment light;

a storage unit configured to store a fluorescence signal of the fluorescence detected by the light detection unit; and

a control unit configured to perform control of at least a start of storing the fluorescent signal by the storage unit,

wherein the control unit is configured to

include a change determination unit for determining whether or not the fluorescence signal detected by the light detection unit has changed beyond at least a predetermined degree of change and a first storage control unit for performing control of at least the start of storing the fluorescence by the storage unit when the change determination unit determines that the fluorescence signal detected by the light detection unit has changed beyond by at least the predetermined degree of change, and

perform control of at least the start of storing the fluorescence signal by the storage unit based on that the fluorescence signal detected by the light detection unit has changed beyond at least the predetermined degree of change, or

wherein the control unit is configured to

include a treatment light determination unit for determining whether or not the light detection unit has detected a signal of the treatment light and a second storage control unit for performing control of the start of storing the fluorescence signal by the storage unit when the treatment light determination unit determines that the light detection unit has detected the signal of the fluorescence light, and

perform control of at least the start of storing the fluorescence signal by the storage unit based on that the light detection unit has detected the signal of the treatment light.

1. A treatment support system comprising:
an irradiation unit configured, in treatment of killing a cancer cell by irradiating a medical agent containing a fluorescent material that has been administered into a body of a subject with treatment light in a predetermined wavelength range, to irradiate the medical agent with the treatment light;
a light detection unit configured to detect, out of the treatment light and fluorescence emitted by the fluorescent material of the medical agent excited by irradiation of the treatment light, at least the fluorescence emitted by the fluorescent material of the medical agent excited by the irradiation of the treatment light;
a storage unit configured to store a fluorescence signal of the fluorescence detected by the light detection unit; and
a control unit configured to perform control of at least a start of storing the fluorescent signal by the storage unit,
wherein the control unit is configured to
include a change determination unit for determining whether or not the fluorescence signal detected by the light detection unit has changed beyond at least a predetermined degree of change and a first storage control unit for performing control of at least the start of storing the fluorescence signal by the storage unit when the change determination unit determines that the fluorescence signal detected by the light detection unit has changed beyond at least the predetermined degree of change, and
perform control of at least the start of storing the fluorescence signal by the storage unit based on that the fluorescence signal detected by the light detection unit has changed beyond at least the predetermined degree of change, or
wherein the control unit is configured to
include a treatment light determination unit for determining whether or not the light detection unit has detected a signal of the treatment light and a second storage control unit for performing control of the start of storing the fluorescence signal by the storage unit when the treatment light determination unit determines that the light detection unit has detected the signal of the treattument light, and
perform control of at least the start of storing the fluorescence signal by the storage unit based on that the light detection unit has detected the signal of the treatment light.

2. The treatment support system as recited in claim 1,
wherein the control unit is configured such that the first storage control unit performs control of at least the start of storing the fluorescence signal by the storage unit based on that fluorescence signal strength detected by the light detection unit has changed beyond at least the predetermined degree of change.

3. The treatment support system as recited in claim 2,
wherein the control unit is configured such that the first storage performs control of at least the start of storing the fluorescence signal by the storage unit based on that a rate of change of the fluorescence signal strength detected by the light detection unit has exceeded a threshold.

4. The treatment support system as recited in claim 2,
wherein the control unit is configured such that the first storage control unit performs control of the start of storing the fluorescence signal by the storage unit based on that the fluorescence signal strength detected by the light detection unit has increased beyond the predetermined degree of change.

5. The treatment support system as recited in claim 4, further comprising:
a display unit configured to display an image indicating a change in the fluorescence signal strength detected by the light detection unit,
wherein the control unit is configured such that, based on that the fluorescence signal strength detected by the light detection unit has increased beyond the predetermined degree of change, the first storage control unit performs control of the start of storing the fluorescence signal by the storage unit and performs control for a start of displaying the image indicating the change in the fluorescence signal strength detected by the light detection unit on the display unit.

6. The treatment support system as recited in claim 4,
wherein the control unit is configured such that the first storage control unit performs control of a stop of storing the fluorescence signal by the storage unit based on that the fluorescence signal strength detected by the light detection unit has decreased beyond a predetermined degree of change.

7. The treatment support system as recited in claim 5,
wherein the control unit is configured such that, based on that the fluorescence signal strength detected by the light detection unit has decreased beyond the predetermined degree of change, the first storage control unit performs control of the stop of storing the fluorescence signal by the storage unit and performs control of the stop of displaying the image indicating the change in the fluorescence signal strength detected by the light detection unit on the display unit.

8. The treatment support system as recited in claim 1,
wherein the light detection unit includes a treatment light detection unit for detecting the treatment light and a fluorescence detection unit for detecting the fluorescence emitted by the fluorescent material of the medical agent excited by the irradiation of the treatment light, and
wherein the control unit is configured such that the first storage control unit performs control of at least the start of storing the fluorescence signal by the storage unit based on that the fluorescence signal detected by the fluorescence detection unit has changed beyond at least the predetermined degree of change, or the second storage control unit performs control of at least the start of storing the fluorescence signal by the storage unit based on that the treatment light detection unit has detected the signal of the treatment light.

9. A treatment support device comprising:
a light detection unit configured, in treatment of killing a cancer cell by irradiating a medical agent containing a fluorescent material that has been administered into a body of a subject with treatment light in a predetermined wavelength range, to detect, out of the treatment light and the fluorescence emitted by the fluorescent material of the medical agent excited by the irradiation of the treatment light, at least the fluorescence emitted by the fluorescent material of the medical agent excited by the irradiation of the treatment light;
a storage unit configured to store a fluorescence signal of the fluorescence detected by the light detection unit; and
a control unit configured to perform control of at least a start of storing the fluorescent signal by the storage unit,
wherein the control unit is configured to
include a change determination unit for determining whether or not the fluorescence signal detected by the light detection unit has changed beyond at least a predetermined degree of change and a first storage control unit for performing control of at least the start of storing the fluorescence by the storage unit when the change determination unit determines that the fluorescence signal detected by the light detection unit has changed beyond by at least the predetermined degree of change, and
perform control of at least the start of storing the fluorescence signal by the storage unit based on that the fluorescence signal detected by the light detection unit has changed beyond at least the predetermined degree of change, or
wherein the control unit is configured to
include a treatment light determination unit for determining whether or not the light detection unit has detected a signal of the treatment light and a second storage control unit for performing control of the start of storing the fluorescence signal by the storage unit when the treatment light determination unit determines that the light detection unit has detected the signal of the treatment light, and
perform control of at least the start of storing the fluorescence signal by the storage unit based on that the light detection unit has detected the signal of the treatment light.
